(19) United States
(12) Patent Application Publication
(10) Pub. No.: US 2026/0148377 A1
(43) Pub. Date: May 28, 2026

(54) Synthetic Scar Augmentation with Granular Control in Magnetic Resonance Imaging

(71) Applicant: Siemens Healthineers AG, Forchheim (DE)

(72) Inventors: Athira Jane Jacob, Plainsboro, NJ (US); Puneet Sharma, Princeton Junction, NJ (US)

(21) Appl. No.: 19/238,604

(22) Filed: Jun. 16, 2025

Related U.S. Application Data
(60) Provisional application No. 63/725,587, filed on Nov. 27, 2024.

(51) Int. Cl. G06T 7/00; G06T 7/11; G06T 7/174; G06T 11/00; G06V 10/774; G16H 10/20

(52) U.S. Cl. CPC G06T 7/0012; G06T 7/11; G06T 7/174; G06T 12/30; G06V 10/774; G16H 10/20; G06T 2207/10088; G06T 2207/30048; G06T 2210/41; G06V 2201/031

(57) ABSTRACT

Parametric control for scar position, size, and/or other characteristic is used to generate a synthetic scar at a location in an MR image. The parameters allow for template-based or other hard-coded generation of textual description of the synthetic scar. The MR image with the synthetic scar, with or without the textural description, may be used for training a DL model for scar detection in MR images. Due to the parametric control, a wide variety of MR images with synthetic scars may be generated, resulting in the DL model as trained being more generalizable.

### BACKGROUND

The present document relates to synthetic scar augmentation for scar detection in magnetic resonance (MR) imaging (MRI). Detection of hyperenhancement (scar tissue enhanced by contrast agent) from cardiac late gadolinium (LGE) MR images is a complex task requiring significant clinical expertise. Enhancement of the scar in the MR image can be subtle and can have varying locations and patterns, which are often not represented adequately in smaller clinical datasets. While deep learning-based (DL) models have shown promising results for LGE analyses, these DL models require large amounts of data with fine-grained annotations. These complexities make developing robust, generalizable automated solutions for LGE detection particularly challenging. The DL model trained on a small LGE dataset may not generalize well.

In one attempt to deal with the data limitations, DL models have been pretrained on large datasets of natural images (no contrast agent) and then fine-tuned on LGE images. The domain shift from natural images to LGE images may result in sub-optimal performance. In another approach, generative DL models, such as generative adversarial networks (GANs) or diffusion models, are used to create LGE images. This presents a chicken and egg problem as there may not be a diverse set of LGE images to train the generative DL model in the first place.

### SUMMARY

Systems, methods, and instructions on computer readable media are provided for generating an MR image with a synthetic scar. Parametric control for scar position, size, and/or other characteristic is used to generate a synthetic scar at a location in an MR image. The parameters allow for template-based or other hard-coded generation of textual description of the synthetic scar. The MR image with the synthetic scar, with or without the textural description, may be used for training a DL model for scar detection in MR images. Due to the parametric control, a wide variety of MR images with synthetic scars may be generated, resulting in the DL model as trained being more generalizable.

In a first aspect, a method is provided for generating a cardiac MR image with a synthetic scar. A first value for a wall location of a scar and a second value for a scar extent in thickness are selected. An image processor forms a region mask from the first value and the second value. The image processor creates the synthetic scar in the region mask and generates the cardiac MR image with the synthetic scar.

In a second aspect, a system of generation of a scar MR image with a synthetic scar is provided. A memory is configured to store multiple value options for a spatial parameter for scar location and to store an input MR image. The multiple options are domain clinical knowledge about scars. An image processor is configured to select a first value of the multiple value options for the spatial parameter, configured to create the synthetic scar at a position based on the selected first value. and configured to generate the scar MR image with the synthetic scar at the position.

In a third aspect, a method is provided for generating a cardiac MR image with a synthetic scar. An image processor granularly controls a spatial position and extent of the synthetic scar relative to a heart wall represented in an input MR image. The image processor creates the synthetic scar and generates the cardiac MR image with the synthetic scar. The synthetic scar located and sized based on the spatial position and extent. The image processor also generates a textual description of the synthetic scar using the spatial position and extent in a template.

Any one or more of the aspects or concepts summarized above or in the Illustrative Embodiments below may be used alone or in combination. The aspects or concepts described for one Illustrative Embodiment or aspect may be used in other embodiments or aspects. The aspects or concepts described for a method or system may be used in others of a system, method, computer program, or non-transitory computer readable storage medium.

These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one implementation of a method for generating a MR image with a synthetic scar;

FIG. 2 illustrates the input MR image and the output MR image with a synthetic scar, scar mask, and textual description;

FIG. 3 illustrates an example implementation of a method for generating a MR image with a synthetic scar;

FIG. 4 shows example MR images with synthetic scars; and

FIG. 5 is a block diagram of one embodiment of a system for generation of synthetic scar images.

### DETAILED DESCRIPTION OF EMBODIMENTS

Synthetic LGE enhancement or scars in MRI are systematically created. A synthetic scar/enhancement is added to normal LGE images. The synthetic scar is granularly and precisely controlled for location and extent or another spatial parameterization. A wide variety of scar distributions is systematically covered. A controller module chooses (e.g., randomly) from the set of values for scar parameters, where the set is established using clinical domain knowledge. Then, a synthetic scar is added to the image based on the chosen value or values. No data-based training is needed for creating MR images with a variety of synthetic scars.

The scar is applied to images with no prior LGE enhancement (actual scarring) but may be added to LGE or other MR images with an existing scar. More than one synthetic scar may be added to an MR image by looping through the creation process. Patterns of LGE enhancement (synthetic scars) can be added. For example, hyperenhancement at right ventricle insertion points is often seen in cases of pulmonary hypertension or sarcoidosis hypertrophic cardiomyopathy. This can be simulated by adding enhancement at and around the RV insertion point landmarks to a series or collection of MR images.

The MR images with synthetic scars may be used to augment training data for DL model training. The limited data of MR images with actual scars is augmented, avoiding over generalization in the DL model training.

Where the DL model is a contrastive language image pre-training (CLIP) or other model with text input, the parameters used by the controller to generate the synthetic scar may be used to generate text as well or instead of the MR image. For example, text descriptions and associated scar masks are created with or without forming an MR image with the synthetic scar. The scar masks may be used as additional training aids for different applications, such as scar segmentation or detection. As another example, the synthetic scar is added to real LGE images, and associated text description is also created. The text description allows utilization of the latest DL methods, such as CLIP-based training, for improving accuracy. The created text can be used to augment a pipeline where clinical reports are used for image-text co-training for classification, detection, segmentation, or another process.

In one implementation, the spatial parameterization used to create the synthetic scar includes the slice level. Any resolution may be used, such as three regions (apical, mid, and basal) regions of the heart. The slice is identified as representing one of the regions. Appending the slice level to the generated text caption allows contextualization of the location of the image for the DL network. In clinical reports, often the text describes the entire patient, while a single image is from a particular location. There is no intuitive way to separate the clinical text to pieces relevant to that particular location. By providing the location of the slice as an additional sentence, the DL model may learn the association implicitly.

As a further example implementation, a DL model is trained to detect myocardial hyperenhancement from LGE images using relevant text from the clinical reports. Due to the limited dataset size and the long-tailed distribution of LGE etiologies, domain knowledge is used to systematically augment the training data with synthetic image-text pairs. During training, the image-text pairs are aligned using global CLIP loss and a local caption loss. The image encoder is initialized with the weights from a myocardial segmentation network. During inference, the trained DL model is queried using the following text: “there is hyperenhancement in the myocardium and there is no hyperenhancement in the myocardium, denoting the positive and negative LGE classes respectively” and an input MR image for the patient.

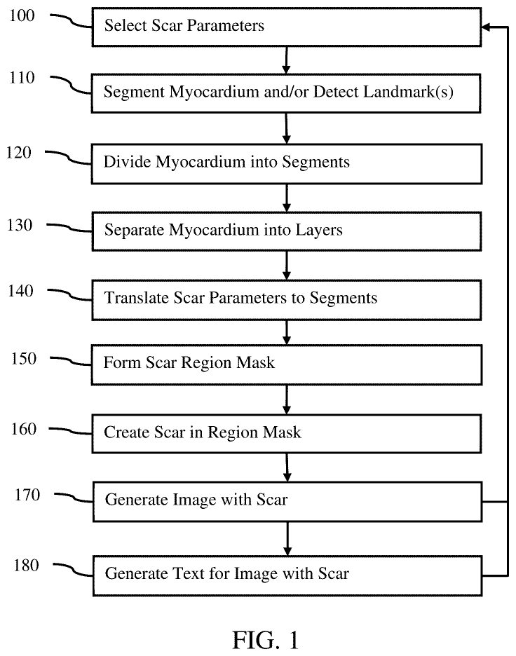

FIGS. 1-5 are directed to generating MR images and/or text for synthetic scars. Data augmentation may be provided for any machine training or another task. After describing the creation of MR images with synthetic scars and/or text describing synthetic scars, the training of an example CLIP-based DL model for scar detection from actual and synthetic training examples is described.

FIG. 1 shows a method for generating a cardiac MR image with a synthetic scar. To provide more examples and/or more evenly distributed examples, including for rarer not-normal situations, multiple MR images with synthetic scar samples are generated for inclusion in training data or for another use. Granular control of spatial parameters is used to generate the synthetic scars. The values of the same parameters may be used to generate text describing the synthetic scars as well.

FIG. 2 shows a generalization of FIG. 1. Actual natural, LGE, or other MR images 200 are input. The output is an MR image 210 with a synthetic scar, a synthetic scar mask 220, and/or text 230 describing the synthetic scar. Synthetic scar/enhancement are added to normal LGE images, while controlling for location and extent. The same input image 200 may be used to generate multiple different samples of synthetic scars. Different input images 200 may be used to create samples (e.g., outputs 210, 220, and/or 230). In one use case, synthetic LGE enhancement samples are created to augment training data for DL model training to detect scars. The text descriptions and scar masks may be used for training the DL model or other models.

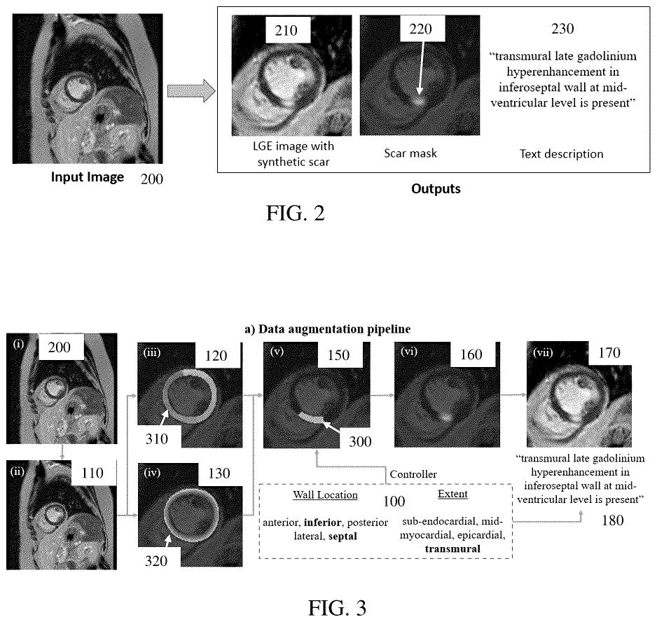

FIG. 3 shows the method for generating MR images with a synthetic scar and associated textual description. In general, FIG. 3 provides MR images illustrating one example implementation of the method of FIG. 1. By repeating performance of the method, the limited data for LGE MR enhancements is augmented with multiple samples. A wide variety of scar distributions may be systematically addressed.

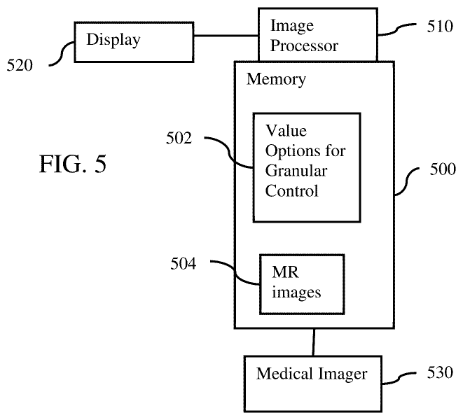

For the methods of FIGS. 1-3, the method is implemented by an image processor (e.g., computer, processor, workstation, or server) using MR images (e.g., LGE MR images) and clinical domain knowledge (e.g., ranges of possible spatial options for scars) stored in memory. For example, the system of FIG. 5 is used. Other systems may be used in other implementations, such as a processor of an MR imaging system performing one or more of the acts.

Additional, different, or fewer acts may be provided. For example, acts 120, 130, and/or 140 are not provided. As another example, only acts 100, 110, and 170 are provided. In yet another example, act 150 is not provided where the scar is created in act 160 without use of the mask. Act 180 may be provided without act 170, or act 170 may be provided without act 180. In yet another example, an act for gathering MR images for input is provided. An act for controlling the selection of act 100 may be provided.

The acts are performed in the order shown or another order. For example, act 100 may be performed after or before any of acts 110, 120, or 130. As another example, acts 120 and 130 may be performed in reverse order. The method is repeated to form additional outputs with synthetic scars.

In act 100, the image processor selects a value for a spatial parameter of the synthetic scar. The selection provides for granular control of the spatial characteristics of the synthetic scar relative to the heart wall (e.g., myocardium) represented in an input MR image. The selection occurs without reference to a particular MR image. The image processor, implementing a controller module, selects a value for each of one or more parameters representing spatial characteristics of the scar. Other characteristics, such as texture and/or intensity, may be used.

Any parameterization of the spatial characteristic of the synthetic scar may be used. In one example, parameters are provided for wall location (e.g., where around the circumference of the myocardium) and/or for scar extent in thickness (e.g., what side and/or where in the thickness of the myocardium). The image processor selects one of anterior, inferior, and posterior and/or one of lateral and septal for the wall location. A combination of the two sets may be used, such as inferoseptal. Other ranges of values or characterizations of wall location may be used, such as angle or segment. The image processor selects one of sub-endocardial, mid-myocardial, epicardial, and transmural for the scar extent. “Transmural” signifies the scar spanning over more than 50% of the myocardial thickness, but other transmural extents may be used. Other ranges of values or characterizations of scar extent may be used.

Slice location is another example parameter. The image processor may select a value for the slice location. For example, the slice may be identified. In a more general approach, the heart or ventricle is divided into two or more ranges, such as three ranges. The slice location is selected as being in one of the ranges, such as mid, apex, or basal ranges. The wall location, scar extent, and slice location are used in one implementation.

The available values or options for each parameter are provided by domain clinical knowledge. The possible scar locations are identified at any resolution and used to make up the available values or options for selection. The range or sets of values is established by expert input or analysis of a study, not by machine-learned selection. This allows for granular control in creating synthetic scars.

The selection may be random. The image processor randomly selects one value for each of the parameters being used. Alternatively, or additionally, the selection follows a search pattern, such as selecting according to a distribution of natural occurrence or selecting in an order over repetition of the method. The user may select in other approaches. The user may select the selection process to use (e.g., random, by natural occurrence, or a combination thereof). The user may limit the options or values available for selection by the image processor, such as to form training data focused on a specific scar area.

Referring to FIG. 3, for every input negative LGE image 200 at a given slice location, a synthetic scar is to be added. Many synthetic samples may be generated by varying input images 200 and/or by varying the selection of values. Random or systematic variation of the values creates different synthetic samples. The distribution of actual samples may not be uniform by pathology or another characteristic. Hence, to be able to train an accurate DL model for scar detection, data augmentation based on synthetic data may be used to generate a more uniform distribution in the training dataset.

In act 150, the image processor forms a region mask 300 from the selected value or values. For example, the region mask is formed from the wall location and scar extent values. The values define an intersection or area where both occur. This area of overlap is the region or scar mask in which the scar is to be located.

Acts 110-140 are example acts performed to provide for forming the scar region mask in act 150. Additional, different, or fewer acts may be used. For example, act 150 is performed without acts 120, 130, and/or 140, such as where the mask is formed from the intersection alone. Act 110 provides the location reference for position on a given MR image. In one approach, acts 120-140 are provided to form the region mask in act 150 relative to standardized heart segments, such as the American Heart Association (AHA) segments.

In act 110, the image processor segments a myocardium as a segmentation from an input MR image 200. One or more landmarks may be detected, such as the right ventricle insertion point (RVIP). Any anatomy to be used as reference for relative placement of the synthetic scar may be detected.

In one approach, two DL networks are used to segment the myocardium and detect anterior and posterior RVIPs on input LGE MRI images 200. Ground truth (GT) annotations for these tasks are relatively easy to create. The myocardium segmentation network may be any DL architecture for segmentation, such as the UNet architecture, and may be trained with any segmentation loss, such as Jaccard Loss. The landmark detection model may also be a fully convolutional image-to-image model trained to predict heatmaps centered on the landmark points. For example, a UNet with a ResNet encoder is used.

In act 150, the image processor identifies the portion of the myocardium for the mask 300. The spatial parameter values are used to identify this portion.

In one approach in act 120, the segmentation of the myocardium is divided into standardized segments 310. The standardized segments may be the AHA segments or other pre-defined segment of heart and/or myocardial regions. For example, the anterior RVIP is used to divide the myocardium into AHA segments. There are four myocardial segments if the slice location is an apical layer and six segments if the slice location is a basal layer or a mid layer.

In act 130, the image processor separates the myocardium (segmentation of the myocardium) into layers 320 along the thickness. For example, the myocardium is a ring (donut) in a cross-sectional slice. This ring is divided into two or more nested or concentric rings, such as three rings (e.g., endocardial, mid-myocardial, and epicardial layers).

The standardized segments correspond to the wall location parameter in one example. The segment or segments that are located at the selected value for wall location account for the layers using the value of the scar extent. The scar extent defines the location along the thickness of the myocardium. The wall location and corresponding segments define the locations around the myocardium. Together, the scar extent value and the wall location value define an area of the myocardium.

In act 140, the scar parameters are translated to the standardized segments. The wall location alone may be used to translate from a portion of the myocardium into the standardized segments. Wall location value (chosen by the controller) is translated into AHA segments through hardcoded values. The slice location may also be used to translate into AHA segments as different segments occur at different portions perpendicular to image slices. For example, inferoseptal on the basal level denotes segment 3 of the AHA segments.

In act 150, the image processor forms the mask region 300 relative to the input MR image 200. The area defined by the intersection of locations of the scar extent and locations of the wall location is the mask region. The identified standardized segments may be used instead of the wall location. The input MR image 200 is the reference as the segmentation and/or landmarks from that image are used to establish the locations for the region mask 300. In one approach, the scar region mask 300 is created using an intersection between the identified AHA segments translated from the chosen wall location and the chosen scar extent. This is the “allowed” region for scar creation.

In act 160, the image processor creates the synthetic scar. The synthetic scar is created in the mask region.

Any scar creation may be used. In one approach, the scar is selected from a collection of templates and positioned randomly within the mask region. The orientation may be random or based on the myocardium at the location.

In another approach, a function is used to create the synthetic scar. The image processor randomly selects a point in the region mask as a center of the synthetic scar. The image processor then randomly selects a size and orientation, if any, of the synthetic scar. The size may be limited, such as using the thickness of the myocardium or region mask at the selected point to limit the size. By using the thickness of the region mask, the thickness of the scar is based on the scar extent value. The thickness of the scar is at or less than the scar extent.

In one implementation, the image processor creates the synthetic scar in the candidate region mask as a randomly placed, oriented, and sized ellipse. Other shapes may be used. A random pixel is chosen from the candidate region as the center of the synthetic scar. The radii of the ellipse are chosen randomly between set minimum and maximum values. The minimum and maximum values are determined as a fraction of the myocardial thickness at that point and/or as a fraction of the scar extent (i.e., depending on whether the scar is chosen to be transmural, or within a specific myocardial layer). Rather than a uniform intensity scar, the created ellipse is smoothed perpendicularly to the circumference with a Gaussian filter for a more natural and continuous appearance.

A further example of this implementation may be represented as:

$${r}_{min} = max ( 0.01 , th * {ρ}_{min} )$$

$${r}_{max} = th * {ρ}_{max}$$

$$r = rand ( {r}_{min} , {r}_{max} , 2 )$$

$$α = rand ( 0 , π )$$

$$σ = rand ( 0 , 1 ) * {s}_{1} + {s}_{2}$$

where, th represents myocardial thickness at that point, 0<ρₘᵢₘ, ρₘₐₓ≤1 are hyperparameters representing ratios relative to myocardial thickness, r∈ [symbol] are the radii of the major and minor axes of the ellipse, α represents the orientation of the major axis of the ellipse relative to the positive x-axis, and σ represents the standard deviation of the Gaussian kernel used for smoothing. The created scar M is min-max normalized to the range of [0, 1].

In one example, λ=0.7, [pmin, pmax]= [0.1, 0.4], [0.3, 0.6], [0.7, 0.1] for single-layer, two-layer, and transmural extents, respectively, and s1=s2=2. These values are selected empirically to ensure image fidelity, anatomical relevance, and/or diversity across generated outputs.

The synthetic scar is the scar mask 220. The scar mask 220 has a position relative to the myocardium of the input MR image 200. Any texture or intensity variation may be used for the synthetic scar.

In act 170, the image processor generates the MR image 210, such as the cardiac MR image or the LGE MR image, with the synthetic scar. The generated MR images 210 with the synthetic scars are synthetic. The samples are images that do not represent an actual patient. Some aspect (e.g., the scar) of the one or more images is made up or different than real for a given patient. The synthetic data does not represent any actual patient collected for the training data but is instead synthesized.

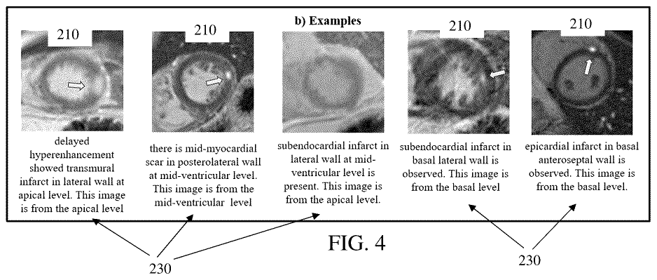

The synthetic scar is located and sized based on the spatial position (e.g., wall location) and extent (e.g., scar extent) selected by the image processor. FIG. 4 shows four examples (first two and last two) with arrows pointing to the synthetic scars. The middle example is an MR image where the scar is located on a different slice. For training, this may be used to learning scar detection where some images do not have a scar.

In one approach for generating the MR image 210 with the synthetic scar, the synthetic scar is overlaid on the input MR image 200. In another approach, the image processor blends the synthetic scar with the input MR image 200. Any blending function may be used. In one example, the scar image M (i.e., synthetic scar at the selected position and orientation) is blended with the image I as,

$${I}_{synth} = I * ( 1 - M ) + γ * max ( I ) * M$$

$$γ = rand ( {b}_{1} , {b}_{2} )$$

where γ controls the brightness of the scar and is randomly chosen between preset minimum and maximum values b₁, b₂. In one example implementation, b₁=0.8, and b₂=1. Other values may be used.

The image processor generates the MR image 210 with the synthetic scar for the selected slice location. The value of the slice location is linked to or associated with the MR image 210.

In act 180, the image processor generates the text 230 describing the synthetic scar. The text is generated, at least in part, from the value or values of one or more of the spatial parameters.

Natural language processing may be used to generate the text 230. In one approach, a large language model is prompted to describe the synthetic scar based on the selected values and outputs the text 230 in response.

In another approach, a template is used. The value or values are input to the template to generate the text 230. For example, the textual description of the synthetic scar uses the spatial position (wall location and/or slice location) and scar extent in a template. Given the chosen scar parameters (e.g., slice location, wall location, and wall or scar extent) from the controller module of act 100, the associated text description is synthesized using one or more preset templates. For example, the template is: “there is <scar extent> delayed enhancement in <wall location> wall. This image is from <slice location> level,” where the words within < > are replaced with the chosen values of the scar parameters. Other templates may be used. To add further variation to the text, the words “delayed enhancement”, “delayed hyperenhancement”, “late enhancement”, “scar”, or “infarct” may be used interchangeably.

The slice location of the input image 200 is appended to stay consistent with real clinical text and contextualize the spatial location of the slice for the model. Alternatively, the slice location is not used.

FIG. 4 shows examples of MR images 210 with synthetic scars and corresponding text 230. In the text 230, the location of the image slice is appended to the caption for additional context. This allows for cases where the scar is present in a different location of the heart (middle image). This is common for actual MR images when the text description is on a patient level (as in a clinical report), while the input image is from a particular location without scarring.

The output (e.g., MR image 210 with the synthetic scar, synthetic scar mask 220, and/or text 230) is added to a database. For example, the output of multiple repetitions of creation of synthetic images is added as augmentation to training data. Alternatively, or additionally, the output is displayed to the user. The display is a visual output. The image processor generates the output. The output may be to a display, into a patient medical record, to a database, and/or to a report.

FIG. 5 shows a system of generation of a scar MR image where the scar is a synthetic scar. The system generates one or more synthetic MR images with synthetic scars. The synthetic scars and corresponding MR images are generated in a controlled manner using granular control in selection of different values of spatial parameters of the scar. Textual description of the synthetic scar may also be created.

The system includes the memory 500, image processor 510, and display 520. The display 520, image processor 510, and memory 500 may be part of the medical imager 530, a computer, server, workstation, or another system for image processing medical images. A workstation or computer without the medical imager 530 and/or display 520 may be used as the system.

Additional, different, or fewer components may be provided. For example, a computer network is included for communication between components. As another example, a user input device (e.g., keyboard, buttons, sliders, dials, trackball, mouse, or other device) is provided for user interaction with creation of synthetic scars, such as by limiting the value options 502 or establishing the search or use pattern of the value options 502.

The system implements the method of FIG. 1, the method of FIG. 2, and/or the method of FIG. 3. Other methods to generate a synthetic scar, MR image with a synthetic scar, and/or text describing a synthetic scar may be implemented by the system.

The medical imager 530 is a MR scanner. For example, the medical imager 530 is a MR system having coils or antennas and an electromagnet around a patient bed. The medical imager 530 is configured by settings to scan a patient. The medical imager 530 is setup to perform a scan for the given clinical problem, such as a cardiac scan. The scan results in scan or image data that may be processed to generate an image of the interior of the patient on the display 520. For example, LGE MR images 504 are formed for input to create synthetic scars, text, or MR images with synthetic scars. In other approaches, the MR images 504 are from a database or mined from patient medical records. The medical imager 530 is not included.

The image processor 510 is a control processor, general processor, digital signal processor, three-dimensional data processor, graphics processing unit, application specific integrated circuit, field programmable gate array, artificial intelligence processor or accelerator, digital circuit, analog circuit, combinations thereof, or other now known or later developed device for processing medical image data. The image processor 510 is a single device, a plurality of devices, or a network. For more than one device, parallel or sequential division of processing may be used. Different devices making up the image processor 510 may perform different functions. In one embodiment, the image processor 510 is a control processor or other processor of a medical diagnostic imaging system, such as the medical imager 530. In alternative embodiments, the image processor 510 is a processor (e.g., server or computer) for generating synthetic data and/or machine training. The image processor 510 operates pursuant to stored instructions, hardware, and/or firmware to perform various acts described herein.

In one embodiment, the image processor 510 is configured to select a value of the multiple value options 502 for a spatial parameter. Multiple value options 502 are provided for each spatial parameter. For example, the spatial parameters include the scar extent in thickness, wall location, and slice location. As another example, the spatial parameters include size, shape, and/or angle. Sets or ranges of values as options 502 are provided for each parameter. The image processor 510 selects one of the values from the set or range for each parameter.

The image processor 510 randomly selects the value from the options 502. Alternatively, the image processor 510 uses a pattern or selection criterion or criteria to select. User input may be used to select.

In one implementation, the image processor 510 is configured to generate a position of the synthetic scar using standardized cardiac segments. The image processor 510 may select a standardized segment from a group of standardized segments. The synthetic scar is then positioned at or in the selected segment. Alternatively, the image processor 510 may translate a selected wall location to standardized segments and use the segment for positioning the synthetic scar.

In another implementation, the image processor 510 is configured to form a region mask based on one or more selected values of the spatial parameters. For example, the wall location and/or slice location are used to form a region mask. The region mask may correspond to or be a standardized segment or segments. A scar extent in thickness may be used to form the region mask, such as limiting the region mask to one or fewer than all concentric layers of the myocardium.

The image processor 510 is configured to create the synthetic scar at a position based on the selected value or values. For example, the synthetic scar is created with random selection of a location in the region mask and random selection of size. Alternatively, the image processor 510 uses the selected value or values to create the synthetic scar. For example, the resolution of the options 502 is relatively high (e.g., within 25% of the image resolution), so that selection of a value corresponds to selection of the spatial characteristic for the synthetic scar without need for further randomization or selection in a region.

The image processor 510 is configured to generate the scar MR image with the synthetic scar at the position. The position identified in the creation of the synthetic scar is known relative to the MR image, such as based on segmentation of the myocardium and/or landmark detection in the MR image. The synthetic scar is positioned and oriented on the MR image. The synthetic scar may be overlaid with or blended with the MR image to generate the scar MR image. For example, the scar mask is blended with the input MR image.

In a further approach, the image processor 510 is configured to generate a text description of the synthetic scar. For example, a template is filled with one or more of the selected values. Where the region mask and/or synthetic scar position uses standardized segments, the text description is generated using one of the standardized cardiac segments. Alternatively, the position of the synthetic scar is translated to the standardized segment for generating the text description. In another alternative, the standardized segment is not used in the text description.

The memory 500 is configured by the image processor 510 or another processor to store information used to create and the created synthetic scars. For example, the memory 500 is configured to store the multiple value options 502 for a spatial parameter for scar location and to store one or more input MR images 504. The multiple options 502 are domain clinical knowledge about scars. The options 502, input MR images 504, metadata for the input MR images 504, selected values, standardized segments, segmentation, detected landmarks, DL model or models, created synthetic scars (i.e., scar masks), created MR images with synthetic scars, created text description of synthetic scars, and/or other information are stored in the memory 500.

The memory 500 is an external storage device, RAM, ROM, database, and/or a local memory (e.g., solid state drive or hard drive). The same or different non-transitory computer readable media may be used for the instructions and other data. The memory 500 may be implemented using a database management system (DBMS) and residing on a memory, such as a hard disk, RAM, or removable media. Alternatively, the memory 500 is internal to the processor 510 (e.g., cache).

The instructions for implementing training, application of a trained DL model, the methods for scar synthesis, and/or the operation discussed herein for the processor 510 are provided on non-transitory computer-readable storage media or memories, such as a cache, buffer, RAM, removable media, hard drive or other computer readable storage media (e.g., the memory 500). Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU or system. Because some of the constituent system components and method steps depicted in the accompanying figures may be implemented in software, the actual connections between the system components (or the process steps) may differ depending upon the manner in which the present embodiments are programmed.

The display 520 is a CRT, LCD, projector, plasma, printer, tablet, smart phone or other now known or later developed display device for displaying information, such as images and/or text. For example, the display 520 displays LGE MR images with synthetic scars, scar masks, and/or text description of the synthetic scar.

In addition to, or an alternative to, display, the created synthetic scar output may be used for training a DL model to detect scars in MR images. An example is described below.

The training data is formed from data of 965 patients with cardiac MRI studies and clinical reports from a single center, of which 404 patients have reported LGE enhancement (scars). The scans were performed on 1.5 T magnets using a T1-weighted, phase sensitive inversion-recovery (PSIR), gradient-echo sequence, and were acquired 10 min after injection of a gadolinium-based contrast agent. The acquisition parameters are as follows, TR/TE: 2.4/1.1 ms; flip angle: 50, slice thickness: 8 mm; in plane resolution between 1.5×1.5 6 mm2 and 2.6×2.6 mm². The patients were divided in train, validation, and testing splits of 772, 91, and 102 patients, respectively, while maintaining class distribution (LGE enhancement presence/absence).

In one implementation, the input MR images 504 are gathered from patient data. For every patient, the segmented PSIR DICOM series is mined using information in their DICOM tags, as this sequence theoretically has the higher spatial resolution desired for LGE detection. This dataset typically has 3 slices, covering apical, mid and basal regions of the heart. Each image may be preprocessed according to the following steps: a) resizing to 1 mm×1 mm resolution, and b) cropping to 112×112 dimension, centered around the LV. The LV mask is obtained from the LGE segmentation. Preprocessing also includes c) Upsampling 2× to 224×224, d) capping intensities at the 98 percentile, and e) normalizing to the range of [0,1].

The DL model is a CLIP model. The CLIP-based training framework consists of a vision encoder and a text encoder to extract features from pairs of images and text, respectively. Each encoder is followed by a set of projection layers to project the features into a common embedding space. More specifically, the input image x_{img} is encoded by the encoder E_{img} into feature vector f_{img}∈Rn. A projection module P_{img} maps the features into the embedding v∈Rp.

$$v = {P}_{img} ( {E}_{img} ( {x}_{img} ) )$$

Similarly, the text encoder Eₜₓₜ encodes the input text into feature vector fₜₓₜ∈Rₘ. A projection module Pₜₓₜ maps the features into the embedding t∈Rp.

$$t = {P}_{txt} ( {E}_{txt} ( {x}_{txt} ) )$$

The similarity vector is calculated using dot product as s=vₙ·tₙ where vₙ, tₙ represent L2 normalized vectors. Then, cross-entropy loss (CE) is used to maximize similarity between image and text embeddings within the same pair and minimize the same across pairs.

The DL model is trained to detect myocardial hyperenhancement from LGE images using relevant text from the clinical reports. Due to the limited dataset size and the long-tailed distribution of LGE etiologies, domain knowledge is used to systematically augment the training data with synthetic image-text pairs. During training, the image-text pairs are aligned using global CLIP loss and a local caption loss. The image encoder is initialized with the weights from a myocardial segmentation network, such as the segmentation network used for the segmentation of act 110. During inference, the trained CLIP-based DL model is queried using the following text: “there is hyperenhancement in the myocardium and there is no hyperenhancement in the myocardium, denoting the positive and negative LGE classes respectively.”

MR images with synthetic scars and the descriptive text are added to the training data, augmenting the training data. The descriptive text uses wall location, scar extent, and slice location values. For example, FIG. 4 shows example text 230 and corresponding MR images 210 with synthetic scars. The MR images without scars of the 965 patients are used as input MR images for forming the MR images with synthetic scars and corresponding text.

Clinical descriptions of LGE use words that describe location relative to the orientation of the LV, defined by the RVIP. The orientation of the LV can vary across patients, and even across images within the same patient. To help the model better associate position descriptors with image features, the orientation of the LV is normalized using the anterior and inferior RVIPs. The RVIPs for each image are obtained from the landmark detection model. Using the two insertion points, each image is rotated to position the line connecting them along the vertical axis of the image, with anterior RVIP being on the top.

CLIP loss aligns image and text embeddings on a global level. However, LGE descriptions from clinical reports are information dense, with multiple words providing critical information about the location, extent, and etiology of the scar. To encourage granular supervision on the level of the individual text tokens, a captioning loss similar to contrastive captioner models is used. A multi-modal decoder is applied to the text tokens, consisting of layers of multi-headed, self-attention layers, followed by cross attention layers attending to features from the vision encoder. The final layer is a classification layer that predicts the distribution of the next token, over the supported vocabulary set.

The vision encoder is initialized with the weights of the trained LGE myocardium segmentation model. Segmenting the LGE myocardium is a closely related task to LGE detection and may aid convergence. As noted below, ablation studies with various other vision encoders may show the effect of this design choice.

In one implementation, the vision encoder is a Densenet 121 (Huang et al. 2017) encoder with UNet decoder, with five downsampling layers. A MaxPool layer is added after the last layer of the DenseNet encoder to get a feature vector size of n=1024. The projection module has two Linear layers, separated by GelU nonlinearity and followed by Dropout and LayerNorm.

The Biomed-BERT is used for the text encoder. The feature vector has size m=768. The text encoder is held frozen for experiments in this study. The projection module optimized end-to-end during training. For each patient, text relevant to LGE imaging is extracted from the respective clinical report, from both the “Findings” and the “Impressions” sections, using a simple keyword search. The extracted text is split into individual sentences (henceforth also referred to as captions), from which one is sampled randomly for every iteration of training. However, this approach introduces an issue: the clinical text annotations are provided at the patient level, whereas the corresponding images represent specific heart sub-regions. To address this, the phrase “This image is from <slice location>level” is appended to each input text, where < > is replaced with basal, mid, or apical, as per the image location. This is done consistently during both training and inference to help the model contextualize the image within the anatomical structure. While limited to three pre-selected slice regions for simplicity, this framework is adaptable to any number of slice regions by adjusting the corresponding section tag.

After training the CLIP-based DL model, the trained model may be compared against a) BiomedCLIP where the same test set, using the same query text used; b) MedFlamingo where the inference query is constructed as a prompt for few shot, visual question-answering using examples: <image 1> Does this cardiac LGE MR image show hyperenhancement in the LV myocardium? Answer: No. <image 2> Does this cardiac LGE MR image show hyperenhancement in the LV myocardium? Answer: Yes. <image 3> Does this cardiac LGE MR image show hyperenhancement in the LV myocardium? Answer:, where image 1, image 2 are example images from the training set, without and with LGE respectively, and image 3 is the query image; and c) Image-only classifier where the model is constructed to be identical to the CLIP-based model, but without the text part (i.e., the image encoder and a projection module followed by a classification head). The image encoder is initialized with the same pretrained weights. The classification head consists of two Linear layers separated by a GeLU non-linearity and a Dropout layer. The baseline model is trained with the GT binary labels extracted from the clinical reports, using binary cross entropy loss.

To reduce the stochastic uncertainty in the results, multiple (n=3) models are trained in every experiment and metrics are averaged. Balanced accuracy is adopted as the main metric to account for class imbalance in the dataset. The Adam optimizer is used with a learning rate of 1e⁻⁴. All models are trained across 4 x NVIDIA A100-SXM4-40 GB GPUs using a batch size of 128, with fully distributed parallel processing. Memory consumption (in GB) and throughput (in FPS) are measured on a single GPU of the same specifications.

The proposed CLIP-based DL model with training data augmented by synthetic scar information obtains a balanced accuracy of 0.83, outperforming the base-lines. Both the publicly available medical VLMs (Biomed-CLIP and MedFlamingo) encounter limitations on this task. These challenges likely stem from two key factors: (a) the VLMs were trained on a diverse array of medical imaging data, with cardiac MR constituting only a small subset, and LGE sequences representing an even smaller fraction of this subset; (b) LGE detection is an inherently challenging task that necessitates specialized clinical domain knowledge and the ability to analyze subtle, fine-grained features within highly localized regions of the images. The image only classifier trained on this dataset demonstrates higher performance but lags behind the proposed CLIP-based DL model with synthetic augmentation by 6 pp.

The impact of omitting different components of the proposed method is explored using ablation studies. Among the components, synthetic scar augmentation has the most significant impact on performance, followed by the normalization of LV orientation, and lastly, the caption loss. As previously described, the proposed model uses a vision encoder pre-trained on the related task of myocardium segmentation in LGE images. Two alternative initializations are: a) task agnostic encoder: This is a unimodal foundation model pretrained on 36 million contrast MR (CMR) images. It was pretrained in a self-supervised manner, without any labeled data, hence is agnostic to any specific task. The model consisted of a ViT-S architecture and was pretrained across many diverse sequences of CMR, such as cine, LGE, and mapping. b) Imagenet pretrained encoder: this uses the same DenseNet-UNet architecture of the proposed model, but with the publicly available ImageNet trained weights. Both initialization choices provide practical alternatives for when training data and/or labels for a directly related task is unavailable. The results indicate that the task-agnostic CMR foundation model (FM), pretrained in a self-supervised manner, outperforms the ImageNet-pretrained model; however, it still falls short compared to the CLIP-based DL model trained on a closely related task.

In a qualitative examination for three patients, patient 1 shows a true positive detection. Though the ground truth text describes LGE presence across the whole heart, the model is able to produce predictions for individual slices, which aids interpretability. Patient 2 presents a false positive, with the network predicting the presence of LGE in apical and mid-ventricular slices. This could be because of the presence of streak artifacts in these two images, degrading image quality and possibly confounding the model. Patient 3 presents a false negative case, with no LGE detected by the model, despite the GT indicating LGE in basal slices. Visual inspection did not reveal LGE in these slices; however, further review of other LGE images in the study confirmed the presence of basal scarring at the level of the LV outflow tract. This highlights a method limitation, as selecting only three input images may omit critical details, reducing the model's efficacy. This is also observed in patient 1, where the selected images do not reflect all the scar present in the description.

Text-based training using clinical reports offers an alternative to obtaining hard labels in clinical domain, which might be expensive and challenging. However, typically used methods such as CLIP, require large amounts of pretraining data, and further finetuning stages for downstream tasks. By incorporating domain knowledge for augmenting, CLIP-based training for small actual datasets is enabled. Synthetic image-text pairs augment the training set, using anatomical information to normalize the orientation of the image, using additional caption loss to enable fine-grained supervision, and using related-task pretraining to improve the accuracy for the task. This text-based training for specific tasks on small datasets, followed by zero-shot inference without any further finetuning stages provides better results.

Listed below are various Illustrative Embodiments. The Illustrative Embodiments summarize different combinations of aspects or features. Other combinations of any of the aspects or features with any other one or more of the aspects or features may be provided. Aspects or features from one type (e.g., method or system) may be used in another type (system or method).

Illustrative Embodiment 1. A method for generating a cardiac magnetic resonance (MR) image with a synthetic scar, the method comprising: selecting a first value for a wall location of a scar and a second value for a scar extent in thickness; forming, by an image processor, a region mask from the first value and the second value; creating, by the image processor, the synthetic scar in the region mask; and generating, by the image processor, the cardiac MR image with the synthetic scar.

Illustrative Embodiment 2. The method of Illustrative Embodiment 1, wherein selecting comprises randomly selecting by the image processor.

Illustrative Embodiment 3. The method of any of Illustrative Embodiments 1-2, wherein selecting comprises selecting based on domain clinical knowledge.

Illustrative Embodiment 4. The method of any of Illustrative Embodiments 1-3, wherein selecting the first value comprises selecting one of anterior, inferior, and posterior and/or one of lateral and septal, wherein selecting the second value comprises selecting one of sub-endocardial, mid-myocardial, epicardial, and transmural.

Illustrative Embodiment 5. The method of any of Illustrative Embodiments 1-4, wherein forming the region mask comprises segmenting a myocardium as a segmentation from an input MR image and identifying a portion of the myocardium from the first value and the second value.

Illustrative Embodiment 6. The method of Illustrative Embodiment 5, wherein identifying comprises dividing the segmentation into standardized segments and translating the first value into the standardized segments, the standardized segments corresponding to the first value being the portion.

Illustrative Embodiment 7. The method of Illustrative Embodiment 6, wherein forming further comprises separating the segmentation into layers, wherein the standardized segments corresponding to the wall location accounts for the layers using the second value.

Illustrative Embodiment 8. The method of any of Illustrative Embodiments 1-7, wherein creating comprises randomly selecting a point in the region mask as a center of the synthetic scar and randomly selecting a size of the synthetic scar.

Illustrative Embodiment 9. The method of Illustrative Embodiment 8, wherein randomly selecting the size comprises randomly selecting the size within a range based on a thickness of the myocardium at the point.

Illustrative Embodiment 10. The method of Illustrative Embodiment 9, wherein the thickness is based on the second value.

Illustrative Embodiment 11. The method of any of Illustrative Embodiments 1-10, wherein forming the region mask comprises forming the region mask relative to an input MR image, and wherein generating the cardiac MR image comprises blending the synthetic scar with the input MR image.

Illustrative Embodiment 12. The method of any of Illustrative Embodiments 1-11, wherein selecting further comprises selecting a third value for a location of a MR slice, and wherein generating the cardiac MR image comprises generating the cardiac MR image of the MR slice of the third value.

Illustrative Embodiment 13. The method of any of Illustrative Embodiments 1-12, further comprising generating text describing the synthetic scar from the first value, the second value, and a template.

Illustrative Embodiment 14. A system of generation of a scar magnetic resonance (MR) image with a synthetic scar, the system comprising: a memory configured to store multiple value options for a spatial parameter for scar location and to store an input MR image, the multiple options comprising domain clinical knowledge about scars; and an image processor configured to select a first value of the multiple value options for the spatial parameter, configured to create the synthetic scar at a position based on the selected first value; and configured to generate the scar MR image with the synthetic scar at the position.

Illustrative Embodiment 15. The system of Illustrative Embodiment 14, wherein the image processor is configured to generate the scar MR image with a scar mask blended with the input MR image.

Illustrative Embodiment 16. The system of any of Illustrative Embodiments 14-15, wherein the image processor is configured to generate a text description of the synthetic scar filling a template with the selected first value.

Illustrative Embodiment 17. The system of Illustrative Embodiment 16, wherein the image processor is configured to select the first value randomly from the multiple value options, to generate a position of the synthetic scar using standardized cardiac segments, and to generate the text description using one of the standardized cardiac segments.

Illustrative Embodiment 18. The system of any of Illustrative Embodiments 14-17, wherein the spatial parameter comprises a scar extent in thickness, a wall location, or a slice location.

Illustrative Embodiment 19. The system of any of Illustrative Embodiments 14-18, wherein the image processor is configured to form a region mask based on the selected first value and to create the synthetic scar with random selection of a location in the region mask and random selection of size.

Illustrative Embodiment 20. A method for generating a cardiac magnetic resonance (MR) image with a synthetic scar, the method comprising: granularly controlling, by an image processor, a spatial position and extent of the synthetic scar relative to a heart wall represented in an input MR image; creating, by the image processor, the synthetic scar; generating, by the image processor, the cardiac MR image with the synthetic scar, the synthetic scar located and sized based on the spatial position and extent; and generating a textual description of the synthetic scar using the spatial position and extent in a template.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A method for generating a cardiac magnetic resonance (MR) image with a synthetic scar, the method comprising:
selecting a first value for a wall location of a scar and a second value for a scar extent in thickness;
forming, by an image processor, a region mask from the first value and the second value;
creating, by the image processor, the synthetic scar in the region mask; and
generating, by the image processor, the cardiac MR image with the synthetic scar.

2. The method of claim 1, wherein selecting comprises randomly selecting by the image processor.

3. The method of claim 1, wherein selecting comprises selecting based on domain clinical knowledge.

4. The method of claim 1, wherein selecting the first value comprises selecting one of anterior, inferior, and posterior and/or one of lateral and septal, wherein selecting the second value comprises selecting one of sub-endocardial, mid-myocardial, epicardial, and transmural.

5. The method of claim 1, wherein forming the region mask comprises segmenting a myocardium as a segmentation from an input MR image, and identifying a portion of the myocardium from the first value and the second value.

6. The method of claim 5, wherein identifying comprises dividing the segmentation into standardized segments and translating the first value into the standardized segments, the standardized segments corresponding to the first value being the portion.

7. The method of claim 6, wherein forming further comprises separating the segmentation into layers, wherein the standardized segments corresponding to the wall location accounts for the layers using the second value.

8. The method of claim 1, wherein creating comprises randomly selecting a point in the region mask as a center of the synthetic scar and randomly selecting a size of the synthetic scar.

9. The method of claim 8, wherein randomly selecting the size comprises randomly selecting the size within a range based on a thickness of the myocardium at the point.

10. The method of claim 9, wherein the thickness is based on the second value.

11. The method of claim 1, wherein forming the region mask comprises forming the region mask relative to an input MR image, and wherein generating the cardiac MR image comprises blending the synthetic scar with the input MR image.

12. The method of claim 1, wherein selecting further comprises selecting a third value for a location of a MR slice, and wherein generating the cardiac MR image comprises generating the cardiac MR image of the MR slice of the third value.

13. The method of claim 1, further comprising generating text describing the synthetic scar from the first value, the second value, and a template.

14. A system of generation of a scar magnetic resonance (MR) image with a synthetic scar, the system comprising:
a memory configured to store multiple value options for a spatial parameter for scar location and to store an input MR image, the multiple options comprising domain clinical knowledge about scars; and
an image processor configured to select a first value of the multiple value options for the spatial parameter, configured to create the synthetic scar at a position based on the selected first value; and configured to generate the scar MR image with the synthetic scar at the position.

15. The system of claim 14, wherein the image processor is configured to generate the scar MR image with a scar mask blended with the input MR image.

16. The system of claim 14, wherein the image processor is configured to generate a text description of the synthetic scar filling a template with the selected first value.

17. The system of claim 16, wherein the image processor is configured to select the first value randomly from the multiple value options, to generate a position of the synthetic scar using standardized cardiac segments, and to generate the text description using one of the standardized cardiac segments.

18. The system of claim 14, wherein the spatial parameter comprises a scar extent in thickness, a wall location, or a slice location.

19. The system of claim 14, wherein the image processor is configured to form a region mask based on the selected first value and to create the synthetic scar with random selection of a location in the region mask and random selection of size.

20. A method for generating a cardiac magnetic resonance (MR) image with a synthetic scar, the method comprising:
granularly controlling, by an image processor, a spatial position and extent of the synthetic scar relative to a heart wall represented in an input MR image;
creating, by the image processor, the synthetic scar;
generating, by the image processor, the cardiac MR image with the synthetic scar, the synthetic scar located and sized based on the spatial position and extent; and
generating, by the image processor, a textual description of the synthetic scar using the spatial position and extent in a template.
